SEQUENCE LISTING (29 sequences)

SEQ ID NO: 1 - RNA, 737 nt, HML.4
gucacugucaagccuccagaugccaucccuuugacuugaaaacucagccaguuuggguggaucaguggccgcucccaaaaaauaagcuggaggcgcuccauaauuuaguccuggaacaguuagaauugggacacauugaggaaucuuucucuccauggaauucacuugucuuuguuauccaaaagaaaucuggggaaaacagagaaugcucacucaucuuagggcaguuaaugcuguacuucaaccucuggggacauuacaaucuggcuuacccucccgcucuaugcucgcugaguauuggccucuaauccucauagaucuuaaagauugcuuuuuuaacauuccacuggccucucaggacuuugaaaaguuugcuuuuauggucccuucccucaacaaugucgcucaggcuacaugcuacuauuggaaaguccuaccacaaggcaugcuuaauagucccacuauuugucaguauuuuguggggcgugugcuucaaccugucagggaucaguuuccccgauguuacaucguucacuacauggaugaucuccucugcacagcccccccauacaccauuuugauuuccugcuuuucugugauucaacaggccauuucagaagcagguuugacuauugcaccagaaaaaauucaaacuaccucucauuuucaauauuugggcaugcaguuggaagacaagcugauuacaccacaaaaaguucagcuuaggagagacgccu

SEQ ID NO: 2 - RNA, 857 nt, HERV.E4.1
ggccaagagauaagaccagcucuggcuaagcgguggccaagagugugggcggaagacaacccuccaggguuggcagucaaccaagcccccgugcuuauagaaguuaagccugggguccagccgguuaggcaaaaacaguacccgguccucagagaagcucuugaagguauccagguccaucucaagugccuaagaaccuuuagaauuauaguuccuugucagucuccauggaacacuccccuccugccuguucccaagccugggaccaaggacuacaggccgguacaggauuugcgcuugguuaaucaggcuacagugacuuuacauccaacaguaccuaaccuguacacauugcugggguugcugccagcugaggacagcugguucaccugcuuggaccugaaagaugccuucuuuagcaucagauuagccccugagagacagaagcuguuugccuuucagugggaagauccagagucaggugucacuacucaauacacuuggacccagccuccccaaagguucaagaacucccccaccaucuuuggggaggcguuggcucgagaccuccagaaguuucccaccagagaccuaggcugcguguugcuccaguacguugaugaccuuuugcugggacaccccacggcagucgggugcgccaagggaacagaugcucuacuccggcaccuggaggacuguggguauaagguguccaagaaaaaaagcucagaucugccgacagcagguauguuacuugggauuuacuauccaacagggggagcacagccugggaucagaaagaaagcaggucauuuguaaucuaccggagccuaagaccagaaggcaggugagagaau

SEQ ID NO: 3 - DNA, 62 bp, Artificial sequence
  Primer
taatacgactcactatagggagatctagcagcatagggggggctgtgcagaggagatcatcc

SEQ ID NO: 4 - DNA, 62 bp, HERV-E4
taatacgactcactatagggagatctagcagcatacagcaaaaggtcatcaacgtactggag

SEQ ID NO: 5 - DNA, 22 bp, HML.4
caacctgtcagggatcagtttc

SEQ ID NO: 6 - DNA, 22 bp, HERV.E4.1
ctcgagacctccagaagtttcc

SEQ ID NO: 7 - RNA, 828 nt, HML-2
cacuguagagccuccuaaacccauaccauuaacuuggaaaacagaaaaacugguguggguaaaucaguggccacuaccaaaacaaaaacuggaggcuuuacauuuauuagcaaaugaacaguuagaaaagggucauauugagccuucauucucgccuuggaauucuccuguguuuguaauucagaagaaaucaggcaaauggcauauguuaacugacuuaagggucguaaacgccguaauucaacccauggggccucuccaacccggguugcccucuccggccaugaucccaaaagauuggccuuuaguuauaauugaucuaaaggauugcuuuuuuaccaucccucuggcggagcaggauugcgaaaaauuugccuuuacuauaccagccauaaauaauaaagaaccagccaccagguuucaguggaaauuguuaccucagggaaugcuuaauaguccaacuauuugucagacuuuuguaggucgagcucuucaaccaguuagagacaaguuuucagacuguuauauuauucauuauauugaugauauuuuaugugcuacagaaacgagagauaaauuaauugacuguuauacauuucugcaagcagagguugccaaugcaggacuggcaauagcaucugauaagauccaaaccucuacuccuuuucauuauuuagggaugcagauagaaaauagaaaaauuaagccacaaaaaauagaaauaagaaaagacacauuaaaaacacuaaaugauuuucaaaaauugcugggagauauuaauuggauucggccaacucuaggcauuccuacuuaugccaugucaaauuu

SEQ ID NO: 8 - RNA, 870 nt, HML-5
uauccaauuuggguagaccaguggccuuuaaagggagagaaauugcaaagagcccaugaguuuguugaagagcaauuaaaagccagccauauagaaccaucaaaaagaccuuggaauucacccauuuucaucauucccaaaaagucugguaaauagagacuuuugcaugacuaacaugcuaucaaugcuaauuugaaaccuaugggaccccuugagcagguguucuccuccccuucagugauuccuugagauuggcauauaauaguuauuggcuuaaaauacugcauuuguacuauucccuuugcagaacaggacagagaaaaauuuguguuuauaauaccagcuauaaauaacgaaaggccagcuccccgauuucacuggaaagugcuuccugaagggaugcuaaacaguccuaccaugugucaguaucauguaaaucaagcuuugcuccucaguagaaaagaauuuccuaauugcaagacuauucauuuuauggaugauauuuuacuagcagccccaauggauccagcacuuuuaaguuuaugugccucuguuguaaagaauacacaguuaagagguuuaaucauagcaccugaaaaaguacagauguccucuccuuggaaauaucuuggguacauacuaacuucguggucaguaagaccucaaaagguuaaauuaaauacuagcaacuuacacaccuuaaaugauuaucagaaauuacuaggcaauauuaacuggcuuugccccaccuugggcauaacuacugauaaguuacagaaccuguuuucuaucuuaaaagacaaugcugcucuagacucucccagguauuuaacuccugcagcacaaagggaaauugaggaaguagagcac

SEQ ID NO: 9 - RNA, 866 nt, HERV-H
cacccuuacccugcucaaugccaauaucccaucccacagcaugcuuuaaaaggauuaaagccuguuaucacucgccugcuacagcaugggcuucuagaaccuauaaacucucuuuucaauucccccauuuuaccuguccaaaaaccggauaagucuuacagacuaguucagaaucugcgucuuaucaaccaaauuguuuugccuauccacccuguggugcccaaccuguacacucuuuuguccucaauaccuuccuccacaacucacuauuccgugcuugaucuuaaagaugguuuuuucacuauucuccugcacuccucgucccagccucucuuugcuuucaccuggacugacccugacacccaucagucccagcagcuuaccuggacugugcugccgcaagguuucagggacagcccucguuacuucagccaagcucuuucucaugaucuacuuuccuuccaccccuccgcuucucaccuuauucaauauauugaugagcuucuucuuuguagccccuccuuugaaucuucucaauaagacacacuucugcuccuucagcauuuauucuccaaaggauaucggguaucccccuccaaagcucaaauuucuucuccauccguuaccuaccucggcauaauucuucauaagaacacacgugcucucccugccgaccgugucugacuaaucucucaagccccaaccccuucuacaaaacaacaacuccuuuccuuccugggcgugguuggauacuuucgccuuuggauaccugguuuugccauccuaacaaaaccauuauauaaacucacaaaaggaaacuuagcugaccccauagauccuaaauccuuuccccacuccucuuucug

SEQ ID NO: 10 - RNA, 773 nt, ERV-9
cugaagcucuuaaaggauuacaggauauuguuaaacauuuaaaagcucaaggcuuaguaaggaaaugcagcagucccugcaacaccccaauucuaggaguacaaaaaccaaauggucacuggagacuagugcaagaucuuagacucaucaaugaggcaguaauuccucuauauccaguuguacccaaccccuauacccugcuuucucaaauaccagaggaagcagaaugguucaugguucuggaccucaaggaugccuucuucuguuuccccugcacucugacucccaguuucuguuugccuuugaggaucccacagaccacacgucccaacuuacauggauggucuugccacaaggguuuagggauagcccucaccuguuuggucaggcacuggcccaagaucuaggccacuucucaaguccaggcacuuugguccuucaguauguggaugauuuacuuuuggcuaccaguucagaagccucaugccagcaggcuacucuagaucucuugaacuuucuaccuaaucaaggguacaaggcaucuaggucaaaggugcagcuuugcuuacagcaggcuaaauaucuaggccuaaucuuagccagagggaccagggcccucagcaaggaaugaauacagccuauacuggcuuauccuugcccuaagacauuaaaacaguugcagggguuccuuggaaucaccggcuuuugccgacuauggaucccuggauacagugagauagucaggccccuccauacucuaaucaaggagaccc

SEQ ID NO: 11 - RNA, 815 nt, HERV-W
gacccaaggcccaacaaggacuccaaaagauuguuaaggaccuaaaagcccaaggccuaguaaaaccaugcaguaaccccugcaguacuccaauuuuaggaguacagaaacccaacagacaguggagguuagugcaagaucucaggauuaucaaugaggcuguuguuccucuauagccagcuguaccuagcccuuauacucugcuuucccaaauaccagaggaagcagagugguuuacaguccuggaccuucaggaugccuucuucugcaucccuguacauccugacucucaauucuuguuugccuuugaagauacuucaaacccaacaucucaacucaccuggacuuuuuaccccaagguucagggauaguccccaucuauuugccaggcauuagcccaagacuugagccaauccucauaccuggacacuugucuucgguagguggaugauuuacuuuuggccgcccauucagaaaccuugugccaucaagccacccaagcgcucuucaauuuccucgcuaccuguggcuacaugguuuccaaaccaaaggcucaacucugcucacagcagguuacuuagggcuaaaauuauccaaaggcaccagggcccucagugaggaacacauccagccuauacuggcuuauccucaucccaaaacccuaaagcaacuaaggggauuccuuggcguaauagguuucugccgaaaauggauucccagguauggcgaaauagccaggucauuaaauacacuaauuaaggaaacucagaaagccaauacccauuuaguaagauggacaacugaaguagaagugg

SEQ ID NO: 12 - RNA, 856 nt, HERV-R
aucauaauccaaugccagucacccuggaacacuccacuuuugcugguacaaaaaccauugccuggaccaggaucugaugaguauauacuggugcagggcuugcaugcuguaaaccaagccacggugaccauacauccaguaguaccaaaccuguauacuuuaaugggacuuauucuagcaagugccaccugguuuacaguccuggacuuaaaggaugcuuuccucugucucuaccuggcaccaguuagucagcccaucuuugcauuuuaaugggacaauucagucacaggcacagggggacagcucgccuggacuagucucccacaaggguucaagaauucucccacaaucuuuggggaagcacuggccucagaccucaaggcauacaccccaccaaaugacaacugcgccuuguugcaguacauagacaacccuucuuuuggcagccccaaccccaagaggacuguaacugggggaacccaggaccuccuccaucucuuaugggaaagcaggguauagaguauccaaaaagaaggcccaaauuugccaugaaaagguuaaauauuuaggcuucauaguaagccaaggggaacgcuggcuuggccauggaugaaagcaagccauuugugcacuuccaacuccaaccaccuggcgccaaauaggggaauucuuaggggcagcaggguucugccauaucuggaucccaaauuucucacuuauagccaggcccuuauaugaagccacaagggagggugaaaaggaaccccuccucugaaaggcugaccagaagaagguguuuaaacaaaucaaagaagcucuaacucaggcuccagccuuaggacugccagauacuacu

SEQ ID NO: 13 - RNA, 819 nt, HERV-L
uuaauggaguuauggcucaggucugacuuacagcagguccagugggucccuggacucauccuguguucauuuucccagugccagaaugcauaauuggcauugucauacuuagaggcuggcagaacccccacauugauucugugacugguaaggugagggcuauuauggugggaaaggccaaauggaagccauuggagcugccuuuaccuaggaaaauaguaaaucaaaaacauuaucaccacccuggagggauugcagagauuagugccaccaucaaggacuugaaaaaugcagggguggcgauucccauaacauccuuguucaacucuccuuuuuggccugugcagaagacagauggaucuuggagaaugagaguggauuaucauaagcuuaaccaaguggugacuccaauugcagcugcuauacaagaugugguuuucauugcucaagcaaauuaauacaucuccugguaccuuguaugcagccauugacuuggcaaauggccuuuuacccauuccauaagccccaccagaagcgauuugccuucagcuggcaaggccagcaauguaucuuuacuguccuacuucagggguauaucaacucuccggcuuugugucauaaucuuauucagagugaucuugaucacuuuucacugccacaagauaucacacugguccauuacauugauggcauuauguugauuggauccaaugagcaagaaguagcaaacacacuggacuuauuggugagacauuugcaugccauaggaugggaaauaaauccaaauaaaauucacgcacccucuaccucaguaaaauu

SEQ ID NO: 14 - DNA, 50 bp, HML-2
aattctaatacgactcactatagggagacacataaaatatcatcaacata

SEQ ID NO: 15 - DNA, 50 bp, HML4
aattctaatacgactcactatagggagatgcagaggagatcatccatgta

SEQ ID NO: 16 - DNA, 50 bp, HML-5
aattctaatacgactcactatagggagactagtaaaatatcatccataaa

SEQ ID NO: 17 - DNA, 50 bp, HERV-H
aattctaatacgactcactatagggagaaaagtagaaggtcatcaatata

SEQ ID NO: 18 - DNA, 51 bp, ERV-9
aattctaatacgactcactatagggagaagtaaatcatccacatactgaag

SEQ ID NO: 19 - DNA, 50 bp, HERV-W
aattctaatacgactcactatagggagaaagtaaatcatccacgtaccga

SEQ ID NO: 20 - DNA, 50 bp, HERV E4.1
aattctaatacgactcactatagggagaccagcaaaaggtcatcaacgta

SEQ ID NO: 21 - DNA, 50 bp, HERV-R
aattctaatacgactcactatagggagaccaaaagaaggttgtctatgta

SEQ ID NO: 22 - DNA, 50 bp, HERV-L
aattctaatacgactcactatagggagatcarcataatgycatcaatgta

SEQ ID NO: 23 - DNA, 24 bp, HML-2
caaccagttagagacaagttttca

SEQ ID NO: 24 - DNA, 23 bp, HML-5
ttgctccccagtagaaaagaatt

SEQ ID NO: 25 - DNA, 25 bp, HERV-H
gacagcccccattacttcagtcaag

SEQ ID NO: 26 - DNA, 22 bp, ERV-9
ccaagatctaggccacttctca

SEQ ID NO: 27 - DNA, 21 bp, HERV-W
catctatttggccaggcatta

SEQ ID NO: 28 - DNA, 20 bp, HERV-R
agacctcaaggcatacaccc

SEQ ID NO: 29 - DNA, 23 bp, HER-L
gctttgtgtcataatcttattcg

(19) United States
(12) Patent Application Publication
(10) Pub. No.: US 2007/0260053 A1
(43) Pub. Date: Nov. 8, 2007

(54) Method for Generating Transcripts

(76) Inventors: Francois Mallet, Villeurbanne (FR); Guy Oriol, Saint-Chamond (FR); Jean-Philippe Pichon, Lyon (FR)

Correspondence Address: OLIFF & BERRIDGE, PLC, P.O. BOX 19928, ALEXANDRIA, VA, 22320, US

(21) Appl. No.: 11/660,952

(22) PCT Filed: Sep. 23, 2005
(86) PCT No.: PCT/FR05/50775

§ 371 Date: Feb. 23, 2007

(30) Foreign Application Priority Data
Oct. 1, 2004 (FR) 0452228

(51) Int. Cl. C07H 21/04; C12N 9/00

(52) U.S. Cl. 536/25.3; 435/183

(57) ABSTRACT

The present invention relates to a method for generating transcripts from: at least one RNA sequence to be amplified comprising a “primer” region and a region of interest, and an amplification primer comprising a promoter region, and a region capable of hybridizing to said “primer” region of the RNA sequence to be amplified, said method being carried out at constant temperature, and comprising the following steps: a) said primer is hybridized with the RNA to be amplified, b) the primer is extended by means of a reverse transcriptase enzymatic activity in order to generate a complementary deoxyribonucleic acid (cDNA) sequence of the RNA to be amplified, c) the RNA to be amplified, hybridized to said cDNA, is cleaved by means of an enzyme that has a ribonuclease H activity, so as to obtain fragments of RNA to be amplified, hybridized to said cDNA, d) the ends of said fragments of RNA to be amplified are extended by means of a reverse transcriptase and strand displacement enzyme, so as to obtain RNA-DNA/DNA hybrids, e) RNA transcripts are obtained from the RNA-DNA/DNA hybrids formed in step d), by means of an enzyme that has an RNA polymerase activity.

The present invention relates to a method for amplifying target nucleic acid sequences.

It is often necessary, in technologies relating to nucleic acids and to genetic material, to determine whether a gene, a part of a gene or a nucleotide sequence is present in a living organism, a cell extract of this organism or any other biological sample. There is a great advantage in searching for specific nucleotide sequences, in particular for detecting pathogenic organisms, determining the presence of alleles, detecting the presence of lesions in a host genome and detecting the presence of a specific mRNA or of the modification of a cellular host. Many genetic diseases can thus be diagnosed by analyzing, and even quantifying, the expression of certain genes. The quantification of the expression of these genes is also fundamental.

Various types of methods for detecting nucleic acids are described in the literature. These methods, particularly those which require the detection of polynucleotides, are based on the pairing properties of the complementary strands of nucleic acids in the DNA-DNA, DNA-RNA and RNA-RNA duplexes through the establishment of hydrogen bonds between the adenine and thymine bases (A-T) and the guanine and cytosine bases (G-C) of double-stranded DNA, or else between the adenine and uracil bases (A-U) in DNA-RNA or RNA-RNA duplexes. The pairing of nucleic acid strands is commonly called “nucleic acid hybridization” or simply “hybridization”.

In general, after having identified the sequence specific for an organism or for a disease that must be analyzed, the nucleic acids must be extracted from a sample and it must be determined whether this sequence (also called “sequence of interest”) is present. Many methods of detection have been developed for this purpose.

The most direct method for detecting the presence of a sequence of interest in a nucleic acid sample is that of obtaining a “probe”, the sequence of which is sufficiently complementary to a part of the target nucleic acid so as to hybridize with the latter. The probe thus synthesized can be brought into contact with a sample containing nucleic acids and, if the sequence of interest is present, the probe will hybridize and will form a reaction product. In the absence of sequence of interest, and with all nonspecific hybridization phenomena being prevented, no reaction product will be formed. If the probe synthesized is coupled to a detectable label, the reaction product can be detected by measuring the amount of label present. Southern blotting (Southern E. M., J. Mol. Biol., 98, 503 (1975)) or Northern blotting or the dot blot technique or sandwich hybridization (DUNN A. R. and HASSEL J. K., Cell, 12, 23 (1977)) constitute examples of methods that can be used.

The main difficulty in this approach is, however, that it is not directly applicable to cases where the number of copies of the sequence of interest present in a sample is low. Under these conditions, it is difficult to distinguish a significant signal greater than the background noise of the reaction (i.e. to distinguish the specific binding of a probe to its sequence of interest from the nonspecific binding between the probe and a sequence other than the sequence of interest). One of the solutions to this problem consists in increasing the detection signal by means of a supplementary reaction. Consequently, various methods have been described in order to increase the detection capacity of these hybridization techniques. These “amplification” methods can be put into three categories: target amplification, proamplification or signal amplification. The articles by firstly, Lewis (1992, Genetic Engineering News 12:1-9) and, secondly, Abramson and Myers (1993, Curer. Opus. Biotechnol. 4:4147) constitute good general reviews of these methods.

Target amplification consists in specifically multiplying a nucleic acid fragment present in a sample. It makes it possible to considerably increase the number of copies of a target nucleic sequence to be detected.

The target amplification techniques described in the literature are based mainly either on the repetition of in vitro DNA synthesis cycles by extension of nucleotide primers hybridized to the target sequence to be amplified, by means of a DNA polymerase (“Polymerase chain reaction”, referred to as PCR: see patents U.S. Pat. No. 4,683,195, U.S. Pat. No. 4,683,202 and U.S. Pat. No. 4,800,159; EP 201 184; the method referred to as “Repair Chain Reaction”, or RCR: see patent application WO 90/01069; the method referred to as “Strand Displacement Amplification” (SDA): see patent EP-0 497 272; the “exonuclease-mediated strand displacement amplification” method: see patent EP 500 224), or on the repetition of in vitro RNA synthesis cycles, by means of a transcription reaction using an RNA polymerase.

Several of these target amplification methods based on the amplification of transcripts have been described. The “TAS” method, described in patent application WO 88/10315, consists of the repetition of a three-step cycle. The first step makes it possible to synthesize a cDNA from RNA in the presence of a reverse transcriptase and of a deoxynucleotide primer containing a sequence specific for a phage RNA polymerase promoter. Following heat denaturation of the RNA/cDNA heteroduplex, the single-stranded cDNA strand is replicated by the reverse transcriptase in the presence of an antisense oligonucleotide primer. The DNA homoduplex thus obtained during this second step contains a double-stranded promoter to which a phage DNA-dependent RNA polymerase can bind. The third step is a transcription, resulting in the production of 30 to 1000 RNA molecules per template, which can, in turn, be used as a template for the synthesis of cDNA and thus to continue the amplification cycle (DAVIS et al., 1990. J. Infect. Dis. 162:13-20).

Various TAS-derived methods exist, including “Self-Sustained Sequence Replication” (or 3SR), described in patent application WO 90/06995 and patent EP 0 373 960, the “Nucleic Acid Sequence-Based Amplification” (or NASBA) method described in patent application WO 91/02818 and European patent EP 329 822, and the “Single Primer Sequence Replication” (or SPSR) method described in patent U.S. Pat. No. 5,194,370.

These methods have in common the combination of three enzymatic activities:

RNA- and DNA-dependent DNA polymerase (reverse transcriptase), ribonuclease H (RNase H, enzyme of Escherichia coli and/or enzymatic activity associated with the reverse transcriptase) and DNA-dependent RNA polymerase (RNA polymerase of the T7 bacteriophage). These methods are based on the same principle and are carried out at fixed temperature (from 37 to 45° C.), according to a continuous process of reverse transcription reactions and transcription reactions in order to replicate an RNA target by means of cDNA. As in the case of TAS, an RNA polymerase (T7 phage) binding site is introduced into the cDNA by means of the primer used for the reverse transcription step. However, the denaturation of the RNA/cDNA heteroduplex is carried out isothermally by specific hydrolysis of the RNA of this heteroduplex by the RNase H activity. The free cDNA is then replicated, using a second oligonucleotide primer, by the reverse transcriptase. The DNA/DNA homoduplex is transcribed to RNA by the T7 RNA polymerase and this RNA can again be used as template for the following cycle.

Another method, referred to as “Ligation Activated Transcription” (or LAT), described in patent U.S. Pat. No. 5,194,370, uses the same enzymatic activities as the 3SR, SPSR and NASBA methods and operates on the same cycle. It differs, however, by virtue of the method of installing a promoter sequence, which, in this case, is introduced onto the end of the cDNA by ligation of a stem-loop structure containing the promoter, in the presence of a DNA ligase.

Two other methods of target amplification based on the amplification of transcripts also exists, described in patent application EP 369 775. One differs from LAT only by virtue of the fact that the promoter is composed of two distinct oligonucleotides. The other uses two enzymatic activities, a ligase and an RNA polymerase that recognizes a double-stranded DNA promoter, capable of extending along an RNA template. The promoter, called mobile promoter, consists of two oligonucleotides. One carries a sense promoter sequence and a probe sequence for the hybridization on the 3′ end of an RNA target, the other carries only an antisense promoter sequence. The 5′ end of the antisense promoter oligonucleotide is juxtaposed, by hybridization, with the 3′ end of the target, and then ligated with this same end. The transcription by means of a suitable RNA polymerase results in the synthesis of multiple transcripts. These transcripts are hybridized and ligated to a second mobile promoter. The transcription of the RNA template results in the synthesis of complementary transcripts. The process can be repeated, resulting in an exponential amplification of the initial target sequence. Mention may also be made of the method described in patent application WO 99/4385, which is a method of aspecific amplification of nucleic acids. This amplification method is also based on transcription, but requires only one primer. The latter, blocked in the 3′ position, allows the addition of a T7 promoter sequence positioned 3′ of a sense RNA. This sequence then allows the transcription of antisense cRNA. This method is nonspecific since the primer used hybridizes to the polyA tail of mRNAs.

In general, amplification methods such as PCR or NASBA require two primers in order to obtain an amplicon. As a result of this, the products of the amplification reaction then themselves become templates for the amplification, making the amplification exponential. While these methods make it possible to obtain very good amplification yields, they remain ill-suited to the simultaneous amplification of several targets (referred to as multiplex amplification), a step which is nevertheless essential when it is desired to analyze an expression profile of several genes. Furthermore, the method described in application WO 99/4385 is based on the amplification of all mRNAs, which can contribute to producing background noise in an analysis of a DNA chip. In addition, this method is limited to the 3′ region of genes. This method is therefore ill-suited to the study of a mutation or of a target region located in the middle of the gene or its 5′ region.

The present invention proposes to solve all the drawbacks of the prior art by providing a specific, linear amplification method which makes it possible to simultaneously amplify target regions of different genes, it being possible for said target regions to be located in the 3′ position, in the 5′ position or in the middle of the gene.

In this respect, the invention relates to a method for generating transcripts from:
an RNA sequence to be amplified comprising a “primer” region and a region of interest, and
an amplification primer comprising a promoter region, and a region capable of hybridizing to said “primer” region of the RNA sequence to be amplified,
   said method being carried out at constant temperature, and comprising the following steps:
a. said primer is hybridized with the RNA to be. amplified,
b. the primer is extended by means of a reverse transcriptase enzymatic activity in order to generate a complementary deoxyribonucleic acid (cDNA) sequence of the RNA to be amplified,
c. the RNA to be amplified, hybridized to said cDNA, is cleaved by means of an enzyme that has a ribonuclease H activity, so as to obtain fragments of RNA to be amplified, hybridized to said cDNA,
d. the ends of said fragments of RNA to be amplified are extended by means of a reverse transcriptase and strand displacement enzyme, so as to obtain RNA-DNA/DNA hybrids,
e. RNA transcripts are obtained from the RNA-DNA/DNA hybrids formed in step d), by means of an enzyme that has an RNA polymerase activity.

For the purpose of the present invention, the term “transcript” is intended to mean a product of transcription, i.e. an RNA newly synthesized during the transcription of the template under the control of the promoter that initiated the transcription.

The term “sequence” is intended to mean a nucleotide sequence containing the natural bases (A, C, G, T if it is a sequence that is deoxyribonucleotide in nature, A, C, G, U if it is a sequence that is ribonucleotide in nature) and/or one or more modified bases, such as inosine, methyl-5-deoxycytidine, deoxyuridine, dimethyl-amino-5-deoxyuridine, diamino-2,6-purine, bromo-5-deoxyuridine or any modified bases that allow a hybridization reaction. The polynucleotide sequence can also be modified in terms of the intemucleotide bonds (possibly involving, for example, phosphorothioate, Hphosphonate, alkyl phosphonate bonds), or in terms of the backbone, as is the case, for example, for alpha-oligonucleotides (French patent No. 2 607 507) or PNAs (EGHOLM et al., 1992, J. Am. Chem. Soc. 114: 1895-1897). In a modified polynucleotide sequence, several modifications as indicated above may be present in combination. This sequence may be of DNA or RNA nature or of DNA/RNA chimera nature, and generally has a “length” of at least 5 deoxyribonucleotides and/or ribonucleotides, that may optionally contain at least one modified nucleotide. A sequence may contain various regions, each associated with various functions.

To carry out the method according to the invention, the starting point is an RNA sequence to be amplified, which could be described as first model of RNA to be amplified. For the purpose of the present invention, the RNA to be amplified may be any ribonucleic acid sequence. Preferably, the RNA to be amplified is a message RNA.

The RNA to be amplified comprises, in particular, a region of interest, i.e. a polynucleotide capable of being hybridized by a hybridization probe specific for said region of interest. This probe thus makes it possible, as described hereinafter, to detect the presence or the absence of the region of interest.

This RNA to be amplified also comprises a “primer” region, i.e. a polynucleotide to which an amplification primer as defined hereinafter can hybridize specifically. Preferably, the “primer” region is located in the 3′ region of the region of interest of the RNA sequence to be amplified.

The expression “sequence or region capable of hybridizing to another sequence/region” is intended to mean a sequence or region that can hybridize to another sequence/region under hybridization conditions which can be determined in each case in a known manner. Reference is also made to complementary sequences/regions. A sequence or region that is strictly complementary to another is a sequence in which each of the bases can pair with a base of the other sequence, without mismatching. The term “hybridization” is intended to mean the process during which, under suitable conditions, two nucleotide fragments that have sufficiently complementary sequences are capable of forming a double strand with stable and specific hydrogen bonds. The hybridization conditions are determined by the stringency, i.e. the strictness of the operating conditions. The higher the stringency on which the hybridization is carried out, the more specific it is. The stringency is defined in particular according to the base composition of a probe/target duplex, and also by the degree of mismatching between two nucleic acids. The stringency can also depend on the reaction parameters, such as the concentration and the type of ionic species present in the hybridization solution, the nature and the concentration of denaturing agents and/or the hybridization temperature. The stringency of the conditions under which a hybridization reaction must be carried out will depend mainly on the hybridization probes used. All these data are well known and the suitable conditions can be determined by those skilled in the art.

For the purpose of the present invention, the term “amplification primer” is intended to mean a sequence which, when it is hybridized to a nucleic acid (of DNA or RNA nature) to be amplified, makes it possible to prime an extrusion reaction. The primer used in the present invention can be divided into two regions, which are, in the 5′-3′ direction:
a promoter region that can be recognized by an RNA polymerase so as to initiate transcription, i.e. the synthesis of an RNA. This promoter region comprises a promoter and a site for transcription initiation by an RNA polymerase.

This promoter region comprises in particular one of the strands of a promoter of an RNA polymerase. By way of example, mention may be made of the natural promoters of RNA polymerases, shortened sequences derived from natural promoters and that have conserved their functionality, or else the “loop” structures capable of initiating transcription as described in MOLLEGAARD N.E. et al. 1994, Proc. Natl. Acad. Sci. USA, 91, 3892-3895. This promoter region may also comprise a sequence favorable to transcription initiation, the function of which is to limit the formation of abortive transcripts. The RNA polymerase may be DNA dependent, which allows the transcription of an RNA from a DNA sequence;
a region capable of hybridizing to the “primer” region of the RNA to be amplified, i.e. complementary to said “primer” region of the RNA to be amplified. This region may be DNA in nature.

Thus, the 5′ end of the primer may comprise nucleotides that do not hybridize with said RNA to be amplified (antisense sequence of a promoter, antisense sequence of a transcription initiation site, etc.), whereas the 3′ end must have a length and a nucleotide composition that allow the initiation of primer extension, under suitable temperature and stringency conditions. According to a preferred embodiment of the invention, the primer is DNA in nature. The primer is not 3′-blocked, which makes it possible to stabilize the DNA-RNA hybrid.

The method according to the invention is an isothermal method, i.e. it does not require any change in temperature during the various steps. According to a preferred embodiment of the invention, the method is carried out at a temperature of between 37° C. and 50° C., preferably between 40 and 45° C., and even more preferably at 41° C.

In step a), said primer is hybridized with the RNA to be amplified at the level of said region allowing the primer/RNA sequence to be amplified hybridization. This hybridization. takes place by complementarity of a predetermined region of the RNA to be amplified with a predetermined region of the primer, under appropriate stringency and temperature conditions. According to a preferred embodiment of the invention, this step is preceded by a step consisting of denaturation of the RNA to be amplified, preferably at 65° C.

In step b), the primer is extended by means of a reverse transcriptase enzymatic activity in order to generate a complementary deoxyribonucleic acid sequence (cDNA) of the RNA to be amplified. This step requires the presence of deoxyribonucleotides, which are used by the enzyme for the synthesis, by complementarity, of the cDNA. This extension is carried out in the 5′-3′ direction of the primer, under suitable stringency and temperature conditions, comparable with those of step a). This reverse transcriptase enzyme is preferably the AMV reverse transcriptase.

In step c), the RNA hybridized to said cDNA is cleaved with an enzyme that has an RNase H activity, so as to obtain fragments of RNA hybridized to said cDNA. The RNAse H activity makes it possible to hydrolize the RNA-DNA hybrids, whereas it does not hydrolyze the RNA-RNA or DNA-DNA hybrids. The RNAse H activity can be obtained by means of a separate enzyme, such as, in particular, by means of the E. coli RNAse H enzyme, or via the RNAse H activity of a reverse transcriptase enzyme, such as, in particular, the AMV reverse transcriptase.

In step d), the ends of said fragments of RNA are extended by means of a reverse transcriptase and strand displacement enzyme, so as to obtain RNA-DNA/DNA complexes. This reverse transcriptase enzyme is preferably the AMV reverse transcriptase.

In step e), RNA transcripts are obtained from the RNA-DNA/DNA complexes formed in step d), by means of an enzyme that has an RNA polymerase activity. The transcription is carried out by synthesis, in the 5′-3′ direction, of an RNA anti-parallel and complementary to the nucleotide strand transcribed.

The enzyme that has an RNA polymerase activity and that is used is an enzyme capable of binding to the promoter of the primer, and of initiating, in vitro, the synthesis of RNA from the transcription initiation site of the primer. According to a preferred embodiment of the invention, the enzyme that has an RNA polymerase activity is a bacteriophage RNA polymerase, preferably the RNA polymerase of the T7 bacteriophage, but other polymerases can be used, such as the RNA polymerase of the bacteriophages T3, SP6, gh-1 etc. Of course, the promoter sequence located on the primer must be suitable for the RNA polymerase used in step e).

The amplification method according to the invention can be carried out by adding, under suitable conditions, all the compounds required for its implementation, i.e. the RNA to be amplified, the enzymes, the amplification primer and the deoxyribonucleotides. This reaction medium is devoid of agents capable of interfering with the amplification process, such as substances which could inhibit the activity of the required enzymes, which could interfere with the hybridization of the primer to the RNA to be amplified, or which could degrade the amplified products.

Thus, according to a preferred embodiment of the invention, steps a) to e) are carried out for a period of time sufficient to obtain a sufficient number of transcripts. Preferably, steps a) to e) are carried out for a period of between 30 min and 3h30, and even more preferably between 1h30 and 2h30.

The transcripts thus obtained can subsequently be detected by any of the techniques known to those skilled in the art.

According to a preferred embodiment of the invention, the method for generating transcripts according to the invention also comprises the following step:
f. a hybridization probe specific for said region of interest is used for detecting the RNA transcripts generated.

The term “detection” is intended to mean either a direct detection by a physical method, or a method of detection using a label. Numerous methods of detection exist for the detection of nucleic acids [see, for example Kricka et al., Clinical Chemistry, 1999, No. 45(4), p. 453-458 or Keller G.H. et al., DNA Probes, 2nd ed., Stockton Press, 1993, sections 5 and 6, p.173-249]. The term “label” is intended to mean a tracer capable of engendering a signal. A nonlimiting list of these traces includes enzymes that produce a signal that can be detected, for example, by colorimetry, fluorescence or luminescence, such as horseradish peroxidase, alkaline phosphatase, beta-galactosidase or glycose-6-phosphate dehydrogenase; chromophores, such as fluorescent, luminescent or dye compounds; electron-dense groups that can be detected by electron microscopy or by virtue of their electrical properties, such as conductivity, by amperometry or voltammetry methods, or by impedance measurements; groups that can be detected by optical methods such as defraction, surface plasmon resonance or contact angle variation, or by physical methods such as atomic force spectroscopy, tunnel effect, etc.; radioactive molecules such as ³²P, ³⁵S or ¹²⁵I. Thus, the polynucleotide can be labeled during the amplification step according to the invention, for example by using a labeled nucleotide triphosphate for the amplification reaction. Indirect systems may also be used, for instance ligands capable of reacting with an anti-ligand. Ligand/anti-ligand couples are well known to those skilled in the art, and this is the case, for example, for the following couples: biotin/streptavidin, hapten/antibody, antigene/antibody, peptide/antibody, sugar/lectin, polynucleotide/sequence complementary to the polynucleotide, successive sequence of histidines, referred to as “tag”, for a metal, for example nickel. In this case, it is the ligand which carries the binding agent. The anti-ligand may be directly detectable by the labels as described in the previous paragraph, or may itself be detectable by a ligand/anti-ligand.

The labeled nucleotide will be a ribonucleotide. The polynucleotide may also be labeled after the amplification step, for example by hybridizing a labeled probe according to the sandwich hybridization technique described in document WO-A-91/19812. Such probes can also be used in an OLISA technique, as described in application WO 03/098217.

This detection can also be carried out using hybridization probes which recognize said regions of interest of the RNAs amplified according to the invention. The term “hybridization probe” is intended to mean a nucleotide fragment comprising from to 100 nucleotide motifs, in particular from 6 to 35 nucleotide motifs, having a hybridization specificity under given conditions for forming a hybridization complex with a region of interest. This hybridization probe may be a “capture” probe. In this case, the target nucleotide fragment can be prelabeled with a label.

The “capture” probe is immobilized or can be immobilized on a solid support by any suitable means, i.e. directly or indirectly, for example by covalence or adsorption. A hybridization reaction is then carried out between said probe and the labeled target nucleotide fragment.

The hybridization probe may also be a “detection” probe. In this case, the hybridization probe may be labeled with a label. A hybridization reaction is then carried out between said detection probe and the target nucleotide fragment. A sandwich assay can also use a capture probe attached to a solid support, to which capture probe the target nucleotide fragment hybridizes, to which target nucleotide fragment the detection probe hybridizes.

Regardless of whether a “capture” probe or a “detection” probe is used, the hybridization reaction can be carried out on a solid support, which includes all the materials on which a nucleic acid can be immobilized. Synthetic materials or natural materials, optionally chemically modified, can be used as a solid support, in particular polysaccharides such as cellulose-based materials, for example paper, cellulose derivatives such as cellulose acetate and nitrocellulose or dextrane, polymers, copolymers, in particular based on styrene-type monomers, natural fibers such as cotton, and synthetic fibers such as nylon; inorganic materials such as silica, quartz, glasses, ceramics; latices; magnetic particles; metal derivatives, gels, etc,. The solid support may be in the form of a microtitration plate, of a membrane as described in the application WO-A-94/12670, of a particle or a biochip. The term “biochip” is intended to mean a solid support that is small in size, on which are attached a multitude of capture probes at predetermined positions. The biochip concept, more specifically the DNA chip concept, dates from the beginning of the 1990s. Nowadays, this concept is being broadened since protein chips are beginning to be developed. It is based on a multidisciplinary technology that integrates microelectronics, nucleic acid chemistry, image analysis and information technology. The operating principle is founded on a basis of molecular biology: the hybridization phenomenon, i.e. the pairing, by complementarity, of the bases of two DNA and/or RNA sequences.

The biochip method is based on the use of capture probes attached to a solid support, on which probes a sample of target nucleotide fragments directly or indirectly labeled with fluorochromes is made to act. The capture probes are positioned in a specific manner on the support or chip and each hybridization gives a specific piece of information, in relation to the target nucleotide fragment. The information obtained is cumulative, and makes it possible, for example, to quantify the level of expression of one or more target genes. In order to analyze the expression of a target gene, a biochip can then be prepared, carrying a very large number of probes which correspond to all or part of the target gene, which is transcribed to mRNA. The complementary DNAs of the mRNAs derived from the target gene(s) that it is desired to analyze are then, for example, hybridized. After hybridization, the support or chip is washed and read, for example, with a scanner and the analysis of the fluorescence is processed by information technology. By way of indication, mention may be made of the DNA chips developed by the company Affymetrix (“Accessing Genetic Information with High-Density DNA arrays”, M. Chee et al., Science, 1996, 274, 610-614. “Light-generated oligonucleotide arrays for rapid DNA sequence analysis”, A. Caviani Pease et al., Proc. Natl. Acad. Sci. USA, 1994, 91, 5022-5026), for molecular diagnosis. In this technology, the capture probes are generally small in size, around twenty nucleotides. Other examples of biochips are given in the publications by G. Ramsay, Nature Biotechnology, 1998, No. 16, p. 40-44; F. Ginot, Human Mutation, 1997, No. 10, p.1-10; J. Cheng et al., Molecular diagnosis, 1996, No. 1(3), p. 183-200; T. Livache et al., Nucleic Acids Research, 1994, No. 22(15), p. 2915-2921; J. Cheng et al., Nature Biotechnology, 1998, No. 16, p. 541-546 or in patents U.S. Pat. No. 4,981,783, U.S. Pat. No. 5,700,637, U.S. Pat. No. 5,445,934, U.S. Pat. No. 5,744,305 and U.S. Pat. No. 5,807,522. Mention may also be made of the low-density chips developed by the company Apibio as described in application WO 03/098217. The main characteristic of the solid support must be that of conserving the characteristics of hybridization of the capture probes to the target nucleic acids while at the same time generating a minimum background noise for the detection method. The present invention is particularly suitable for these low-density chip systems.

The method according to. the invention can be carried out in order to generate RNA transcripts originating from various genes. It is even one of the advantages of the present invention that it is possible to simultaneously amplify target regions of different genes, it being possible for said target regions to be located in the 3′ position, in the 5′ position or in the middle of the gene.

In this respect, the invention also relates to a method for generating transcripts from:
at least a first RNA sequence to be amplified comprising a “primer” region and a region of interest, and a first amplification primer comprising a promoter region and a region capable of hybridizing to said “primer” region of the first RNA sequence to be amplified,
at least a second RNA sequence to be amplified, different than said first RNA sequence to be amplified, and comprising a “primer” region and a region of interest, and a second amplification primer comprising a promoter region and a region capable of hybridizing to said “primer” region of the second RNA sequence to be amplified,
   said method being carried out at constant temperature, and comprising the following steps:
a). said first and second primers are hybridized with said first and second RNAs to be amplified,
b). said first and second primers are extended by means of a reverse transcriptase enzymatic activity in order to generate a first complementary deoxyribonucleic acid (cDNA) sequence of the first RNA to be amplified and a second complementary deoxyribonucleic acid (cDNA) sequence of the second RNA to be amplified,
c). the first and second RNAs to be amplified, hybridized respectively to the first and second cDNAs, are cleaved by means of an enzyme that has a ribonuclease H activity, so as to obtain first fragments of RNA to be amplified, hybridized to the first cDNA, and second fragments of RNA to be amplified, hybridized to the second cDNA,
d). the ends of said first and second fragments of RNA to be amplified are extended by means of a reverse transcriptase and strand displacement enzyme, so as to obtain RNA-DNA/DNA hybrids,
e). transcripts of said first and second RNAs to be amplified are obtained from the RNA-DNA/DNA hybrids formed in step d), by means of an enzyme that has an RNA polymerase activity.

This method can be used in a comparable manner for generating transcripts from RNA to be amplified originating from 3, 4, 5, 10 or more different genes. Steps a) to e) are performed in a manner comparable to those described above, and are carried out for a period of time sufficient to obtain a sufficient number of transcripts, as defined above.

For detecting the transcripts, use is preferably made, in a step f), of a first hybridization probe for detecting the transcripts of the first RNA to be amplified and a second hybridization probe for detecting the transcripts of the second RNA to be amplified.

Preferably, and as defined above, the enzyme that has an RNA polymerase activity is a bacteriophage RNA polymerase, and even more preferably the RNA polymerase of the T7 bacteriophage.

The attached figures are given by way of explanatory example and are in no way limiting in nature. They will make it possible to understand the invention more clearly.

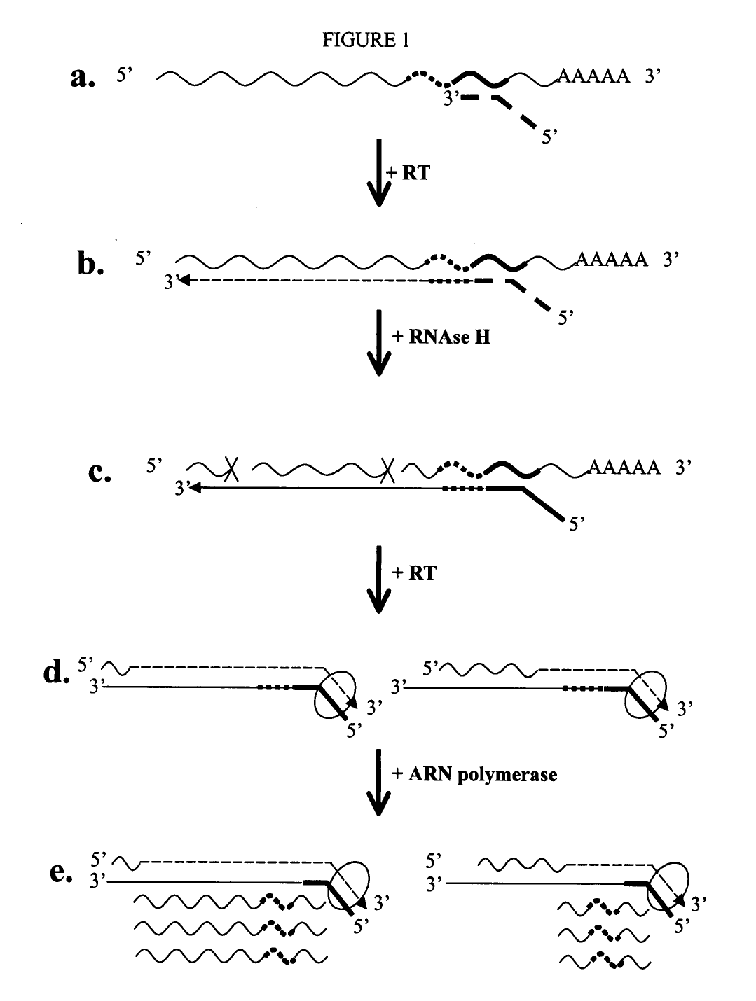

FIG. 1 represents schematically the steps of the method according to the invention.

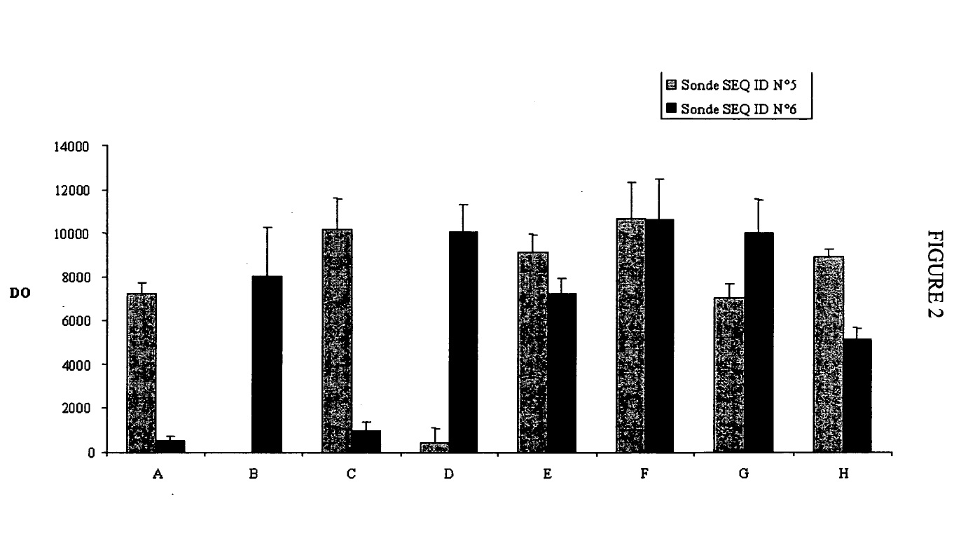

FIG. 2 represents the signal emitted by a hybridization probe hybridized to transcripts obtained according to the invention and as described in example 2.

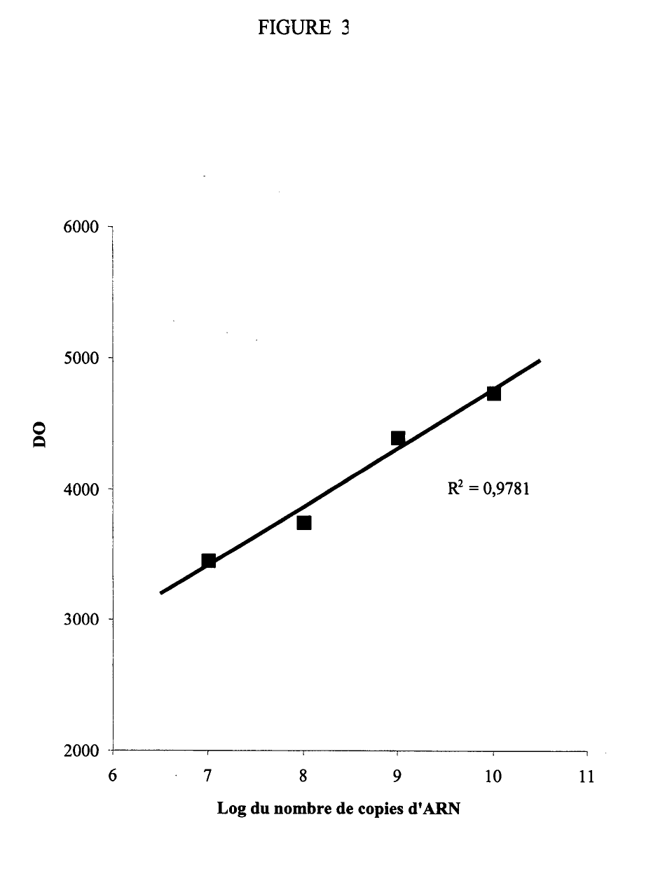

FIG. 3 represents the linear regression obtained after amplification of a range of transcripts (1×10⁷, 1×10⁸ , 1×10⁹ and 1×10¹⁰ copies) according to the protocol described in example 3.

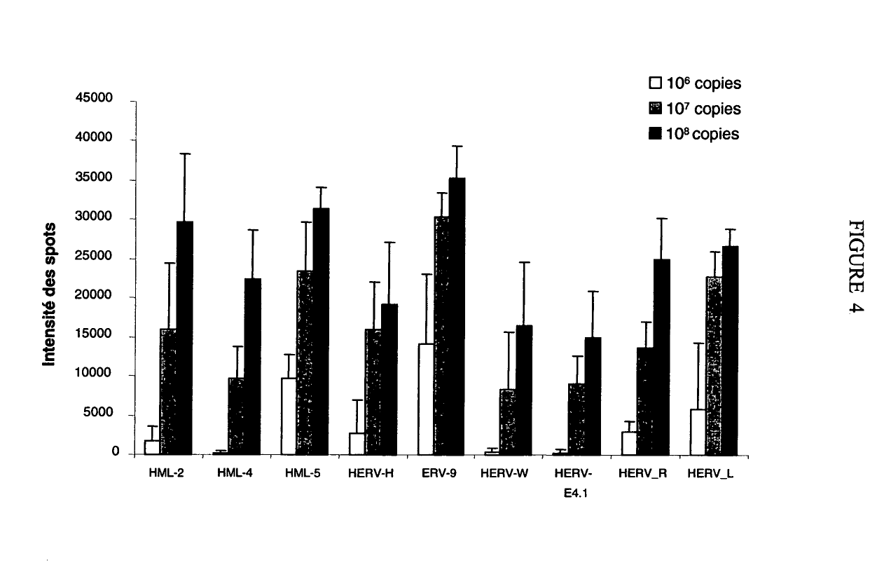

FIG. 4 represents the signal emitted (averaged over 3 replicates) and its standard deviation with respect to each specific probe after hybridization and visualization of the amplification products obtained according to the protocol described in example 4.

In FIG. 1, the sequences of ribonucleic nature are represented as a wavy line, whereas the sequences of deoxyribonucleic nature are represented as a straight line.

In step a), the primer of DNA nature (represented by a long dashed line) is hybridized to the “primer” region (represented by a bold line) of the template RNA, the promoter sequence in the 5′ position of the primer not hybridizing to the template RNA. To understand the invention more clearly, the sequence of interest that it is desired to detect after the amplification phase is represented as a short dashed line, on the 5′ side of the “primer” region. However, the sequence of interest and the “primer” sequence may be one and the same sequence.

In step b) an enzyme that has a reverse transcriptase (RT) activity is made to act in order to extend, in the 5′-3′ direction, the primer hybridized to the template RNA. A DNA (cDNA, represented as a dashed line), the sequence of which is complementary to that of the template RNA, is obtained. A template RNA/cDNA double-stranded hybrid sequence is thus obtained between the target region and the 5′ end of the template RNA. By virtue of their lack of complementarity, the promoter sequence of the primer, and the sequence located on the 3′ side of the target region of the target RNA, remain nonhybridized (single-stranded) sequences.

In step c), an enzyme that has an RNAse H activity is made to act so as to induce cleavages within the template RNA. These cleavages are nonspecific and make it possible to release the 3′ OH end (represented by a cross) of fragments of RNA, hybridized to the cDNA.

The 3′ OH ends thus released are extended, in step d), by means of an enzyme that has a reverse transcriptase activity and a strand displacement activity. The extension ends with the synthesis of the strand complementary to the promoter sequence of the primer. Various RNA-DNA/DNA double-stranded hybrid sequences are thus obtained, comprising a double-stranded promoter region (represented by a circle) capable, under the action of a DNA-dependent RNA polymerase, of initiating a transcription. Two different hybrids, obtained from two different cleavage sites, are thus represented by way of indication. The action of a DNA-dependent RNA polymerase enzyme makes it possible to obtain, in step e), RNA transcripts corresponding to the various hybrids obtained in step d). A population of transcripts of different size, but all comprising the region of interest that it is desired to detect, is thus obtained.

The following examples are given by way of illustration and are in no way limiting in nature. They will make it possible to understand the invention more clearly.

### EXAMPLE 1

### Obtaining RNA Sequences to be Amplified

A first RNA sequence to be amplified, corresponding to SEQ ID No. 1:

GUCACUGUCA AGCCUCCAGA UGCCAUCCCU UUGACUUGAA
AACUCAGCCA GUUUGGGUGG AUCAGUGGCC GCUCCCAAAA
AAUAAGCUGG AGGCGCUCCA UAAUUUAGUC CUGGAACAGU
UAGAAUUGGG ACACAUUGAG GAAUCUUUCU CUCCAUGGAA
UUCACUUGUC UUUGUUAUCC AAAAGAAAUC UGGGGAAAAC
AGAGAAUGCU CACUCAUCUU AGGGCAGUUA AUGCUGUACU
UCAACCUCUG GGGACAUUAC AAUCUGGCUU ACCCUCCCGC
UCUAUGCUCG CUGAGUAUUG GCCUCUAAUC CUCAUAGAUC
UUAAAGAUUG CUUUUUUAAC AUUCCACUGG CCUCUCAGGA
CUUUGAAAAG UUUGCUUUUA UGGUCCCUUC CCUCAACAAU
GUCGCUCAGG CUACAUGCUA CUAUUGGAAA GUCCUACCAC
AAGGCAUGCU UAAUAGUCCC ACUAUUUGUC AGUAUUUUGU
GGGGCGUGUG CUUCAACCUG UCAGGGAUCA GUUUCCCCGA
UGUUACAUCG UUCACUACAU GGAUGAUCUC CUCUGCACAG
CCCCCCCAUA CACCAUUUUG AUUUCCUGCU UUUCUGUGAU
UCAACAGGCC AUUUCAGAAG CAGGUUUGAC UAUUGCACCA
GAAAAAAUUC AAACUACCUC UCAUUUUCAA UAUUUGGGCA
UGCAGUUGGA AGACAAGCUG AUUACACCAC AAAAAGUUCA
GCUUAGGAGA GACGCCU

was produced from cloned sequences derived from a region of the pol gene of the HML-4 endogenous retrovirus, in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

In a comparable manner, a second RNA sequence to be amplified, of SEQ ID No. 2:

GGCCAAGAGA UAAGACCAG CUCUGGCUAA GCGGUGGCCA
AGAGUGUGGG CGGAAGACAA CCCUCCAGGG UUGGCAGUCA
ACCAAGCCCC CGUGCUUAUA GAAGUUAAGC CUGGGGUCCA
GCCGGUUAGG CAAAAACAGU ACCCGGUCCU CAGAGAAGCU
CUUGAAGGUA UCCAGGUCCA UCUCAAGUGC CUAAGAACCU
UUAGAAUUAU AGUUCCUUGU CAGUCUCCAU GGAACACUCC
CCUCCUGCCU GUUCCCAAGC CUGGGACCAA GGACUACAGG
CCGGUACAGG AUUUGCGCUU GGUUAAUCAG GCUACAGUGA
CUUUACAUCC AACAGUACCU AACCUGUACA CAUUGCUGGG
GUUGCUGCCA GCUGAGGACA GCUGGUUCAC CUGCUUGGAC
CUGAAAGAUG CCUUCUUUAG CAUCAGAUUA GCCCCUGAGA
GACAGAAGCU GUUUGCCUUU CAGUGGGAAG AUCCAGAGUC
AGGUGUCACU ACUCAAUACA CUUGGACCCA GCCUCCCCAA
AGGUUCAAGA ACUCCCCCAC CAUCUUUGGG GAGGCGUUGG
CUCGAGACCU CCAGAAGUUU CCCACCAGAG ACCUAGGCUG
CGUGUUGCUC CAGUACGUUG AUGACCUUUU GCUGGGACAC
CCCACGGCAG UCGGGUGCGC CAAGGGAACA GAUGCUCUAC
UCCGGCACCU GGAGGACUGU GGGUAUAAGG UGUCCAAGAA
AAAAAGCUCA GAUCUGCCGA CAGCAGGUAU GUUACUUGGG
AUUUACUAUC CAACAGGGGG AGCACAGCCU GGGAUCAGAA
AGAAAGCAGG UCAUUUGUAA UCUACCGGAG CCUAAGACCA
GAAGGCAGGU GAGAGAAU

was produced from cloned sequences derived from a region of the pol gene of the HERV-E4.1 endogenous retrovirus.

### EXAMPLE 2

### Generation of Transcripts by Means of the Method According to the Invention, From RNA Sequences as Defmed in Example 1

For amplifying the RNA sequence SEQ ID No. 1, use was made of a primer sequence of SEQ ID No. 3:

SEQ ID N^{o} 3:
TAATACGACT CACTATAGGG AGATCTAGCA GCATAGGGGG
GGCTGTGCAG AGGAGATCAT CC

comprising
a promoter region comprising a T7 RNA polymerase promoter (indicated in italics on SEQ ID No. 3) and a transcription initiation site (indicated in bold on SEQ ID No. 3),
a region capable of hybridizing to a primer region of the RNA sequence to be amplified (indicated as underlined on SEQ ID No. 3).

This primer was non-3′-blocked.

It should be noted that the region capable of hybridizing to the primer region may be shorter, and may comprise in particular from 15 to 30 base pairs. It should also be noted that, included between the initiation site and the region capable of hybridizing to the primer region, is a sequence favoring the initiation, which limits the formation of abortive transcripts.

Amplifying the RNA sequence of SEQ ID No.2, use was made of a primer sequence of SEQ ID No. 4:

SEQ ID N^{o} 4:
TAATACGACT CACTATAGGG AGATCTAGCA GCATACAGCA
AAAGGTCATC AACGTACTGG AG

comprising
a promoter region comprising a T7 RNA polymerase promoter (indicated in italics on SEQ ID No. 4) and a transcription initiation site (indicated in bold on SEQ ID No. 4),
a region capable of hybridizing to a primer region of the RNA sequence to be amplified (indicated as underlined on SEQ ID No. 4).

This primer was non-3′-blocked.

It should be noted that the region capable of hybridizing to the primer region may be shorter, and may comprise in particular from 15 to 30 base pairs. It should also be noted that, included between the initiation site and the region capable of hybridizing to the primer region, is a sequence favoring initiation, which limits the formation of abortive transcripts.

The generation of the transcript was carried out in a reaction mixture (NucliSens Basic Kit, bioMérieux) comprising an enzyme that has a reverse transcriptase activity (AMV-RT), an enzyme that has an RNaseH activity (RNase H) and an enzyme that has an RNA polymerase activity (T7 RNA polymerase).

The amplification was carried out in the presence of:
the primer sequence of SEQ ID No. 3 (0.5 μM) for amplifying the RNA sequence of SEQ ID No. 1,
the primer sequence of SEQ ID No. 4 (0.5 μM) for amplifying the RNA sequence of SEQ ID No. 2,
the primer sequence of SEQ ID No. 3 (0.5 μM) and the primer sequence of SEQ ID No. 4 (0.5 μM) for simultaneously amplifying the RNA sequence of SEQ ID No. 1 and the RNA sequence of SEQ ID No. 2.

Thus, the amplification of the RNA sequence SEQ ID No. 1 and the amplification of the RNA sequence SEQ ID No. 2, with incorporation of biotin, were carried out:
independently of one another:

For this, a known amount of RNA of SEQ ID No. 1 or of RNA of SEQ ID No. 2 in an aqueous solution (1×10⁹ or 1×10¹⁰ copies in 5 μl H₂O) is mixed with 10 μl of reaction mixture (NucliSens Basic Kit), to which UTP-biotin (0.6 mM) and the primer for amplifying SEQ ID No. 1 or SEQ ID No. 2 have been added beforehand.
simultaneously:

For this, a known amount of RNA of SEQ ID No. 1 and of RNA of SEQ ID No. 2 in an aqueous solution were mixed with 10 μl of reaction mixture (NucliSens Basic Kit), to which UTP-biotin and the primers for amplifying SEQ ID No. 1 and SEQ ID No. 2 have been added beforehand.

For each of the amplification reactions, the resulting volume was 15 μl (qs nuclease-free H₂O). The mixture was subsequently incubated for 5 minutes at 65° C., then for 5 mins at 41° C. in order to allow the hybridization of the primer to the RNA to be amplified.

A volume of 5 μl of enzyme mixture (NucliSens Basic Kit) was subsequently added. The mixture, 20 μl in volume, was incubated for 90 min at 41° C. so as to allow:
1. hybridization of the primer with the RNA to be amplified,
2. extension of the primer by means of the reverse transcriptase enzymatic activity in order to generate a complementary deoxyribonucleic acid (cDNA) sequence of the RNA to be amplified,
3. cleavage of the RNA to be amplified, hybridized to said cDNA, by means of the enzyme that has ribonuclease H activity, so as to obtain fragments of RNA to be amplified, hybridized to said cDNA,
4. extension of the ends of said fragments of RNA to be amplified, by means of the reverse transcriptase and strand displacement enzyme, so as to obtain RNA-DNA/DNA hybrids,
5. production of the RNA transcripts from the RNA-DNA/DNA hybrids formed in step d), by means of the enzyme that has an RNA polymerase activity.

The RNA transcripts obtained were analyzed by the ELOSA method (Mallet F. et al., 1993). This method allows the specific detection of nucleic acid sequences by means of the hybridization of the latter to oligonucleotide probes (called detection probes) grafted onto the bottom of microtitration plate wells.

The probe for demonstrating SEQ ID No. 1 was the sequence SEQ ID No. 5: CAACCTGTCAGGGATCAGTTTC.

The probe for demonstrating SEQ ID No. 2 was the sequence SEQ ID No. 6: CTCGAGACCTCCAGAAGTTTCC.

The labeling of the RNA transcripts with biotin made it possible to visualize the hybridization by virtue of the action of a streptavidin-enzyme conjugate and of the corresponding substrate, and the ODs were read by spectrometry. The OD was proportional to the amount of transcripts generated by the method according to the invention.

The results obtained are shown in FIG. 1. The letters A to D corresponded to conditions for amplifying SEQ ID No. 1 and SEQ ID No. 2 independently of one another (A: 1×10⁹ copies of SEQ ID No. 1; B: 1×10⁹ copies of SEQ ID No. 2; C: 1×10¹⁰ copies of SEQ ID No. 1; D: 1×10¹⁰ copies of SEQ ID No. 2), whereas the letters E to H corresponded to amplifications of SEQ ID No. 1 and SEQ ID No. 2 simultaneously (E: 1×10⁹ copies of SEQ ID No. 1 and 1×10⁹ copies of SEQ ID No. 2; F: 1×10¹⁰ copies of SEQ ID No. 1 and 1×10¹⁰ copies of SEQ ID No. 2; G:1×10⁹ copies of SEQ ID No. 1 and 1×10¹⁰ copies of SEQ IDNo.2; H: 1×10¹⁰ copies of SEQ ID No. 1 and 1×10⁹ copies of SEQ ID No. 2). The transcripts obtained were detected by means of the presence of a detection probe of SEQ ID No. 5 (gray columns) or by means of the presence of a detection probe of SEQ ID No. 6 (black columns).

The results show that the signals detected were comparable between the independent amplifications and the amplifications carried out in duplexes. Thus, the number of transcripts generated was comparable whether the RNA to be amplified has a SEQ ID No. 1 or a SEQ ID No. 2 (the OD read was comparable when amplification was according to condition A and condition B). Furthermore, the number of transcripts generated was greater when the amount of RNA to be amplified was increased (the OD read was lower during the amplification according to condition A than during that according to condition C; similarly, the OD read was lower during the amplification according to condition B than during that according to condition D).

Finally, the number of transcripts generated during the simultaneous amplification of SEQ ID No. 1 and SEQ ID No. 2 was comparable to the number of transcripts generated during the amplification of SEQ ID No. 1 and SEQ ID No. 2 independently of one another. Even when the simultaneous amplification is carried out on amounts of transcripts that differ by one log, the signals remain identical to those obtained during the amplification of SEQ ID No. 1 and SEQ ID No. 2 independently of one another.

Comparable results were obtained by analyzing the transcripts generated on an OLISA chip. OLISA chips are low-density DNA chips (up to 128 probes per chip) arranged on a standard 96-well plate format. The oligonucleotide detection probes, distributed in crowns, are grafted onto the well bottom. The colorimetric visualization of the probe/target hybrids is carried out using a streptavidin-enzyme conjugate that involves biotin labeling of the target.

An additional RNase-digestion step confirmed that the product detected is indeed a cRNA.

These results demonstrate that the method according to the invention is therefore particularly suitable for amplifying transcripts for the purpose of quantitatively analyzing them on a DNA chip. This is because the fact that this method is transcriptional and that the amplification products never become templates for the amplification confers on this amplification a linear nature that is entirely suited to quantifying transcripts on a biochip.

### EXPERIMENT 3

### Evaluation of the Linearity of the Amplification of the Method According to the Invention

This example is carried out based on SEQ ID No. 2 used as RNA sequence to be amplified and SEQ ID No. 4 used as amplification primer.

The method for generating the transcripts is carried out according to the conditions described in example 2. In this case, the primer of SEQ ID No. 2 is used at a concentration of 0.5 μM. A range of transcripts (1×10⁷, 1×10⁸, 1×10⁹ and 1×10¹⁰ copies) is amplified and the reaction products are analyzed on an OLISA chip using the probe of SEQ ID No. 6, according to a principle comparable to that developed in example 2.

The linear regression produced from the means obtained for at least 4 measurements of each point results in a correlation coefficient straight line R²=0.9781 being obtained as represented in FIG. 3. This therefore attests to the linearity of the amplification of the method according to the invention.

### EXPERIMENT 4

### Multiplex Amplification

For this experiment, the amplification protocol was carried out as previously described in examples 1 and 2.

Thus, a first RNA sequence to be amplified, corresponding to SEQ ID No. 7:

CACUGUAGAG CCUCCUAAAC CCAUACCAUU AACUUGGAAA
ACAGAAAAAC UGGUGUGGGU AAAUCAGUGG CCACUACCAA
AACAAAAACU GGAGGCUUUA CAUUUAUUAG CAAAUGAACA
GUUAGAAAAG GGUCAUAUUG AGCCUUCAUU CUCGCCUUGG
AAUUCUCCUG UGUUUGUAAU UCAGAAGAAA UCAGGCAAAU
GGCAUAUGUU AACUGACUUA AGGGUCGUAA ACGCCGUAAU
UCAACCCAUG GGGCCUCUCC AACCCGGGUU GCCCUCUCCG
GCCAUGAUCC CAAAAGAUUG GCCUUUAGUU AUAAUUGAUC
UAAAGGAUUG CUUUUUUACC AUCCCUCUGG CGGAGCAGGA
UUGCGAAAAA UUUGCCUUUA CUAUACCAGC CAUAAAUAAU
AAAGAACCAG CCACCAGGUU UCAGUGGAAA UUGUUACCUC
AGGGAAUGCU UAAUAGUCCA ACUAUUUGUC AGACUUUUGU
AGGUCGAGCU CUUCAACCAG UUAGAGACAA GUUUUCAGAC
UGUUAUAUUA UUCAUUAUAU UGAUGAUAUU UUAUGUGCUA
CAGAAACGAG AGAUAAAUUA AUUGACUGUU AUACAUUUCU
GCAAGCAGAG GUUGCCAAUG CAGGACUGGC AAUAGCAUCU
GAUAAGAUCC AAACCUCUAC UCCUUUUCAU UAUUUAGGGA
UGCAGAUAGA AAAUAGAAAA AUUAAGCCAC AAAAAAUAGA
AAUAAGAAAA GACACAUUAA AAACACUAAA UGAUUUUCAA
AAAUUGCUGG GAGAUAUUAA UUGGAUUCGG CCAACUCUAG
GCAUUCCUAC UUAUGCCAUG UCAAAUUU

was produced from cloned sequences derived from a region of. the pol gene of the HML-2 endogenous retrovirus in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

In a comparable manner, a second RNA sequence to be amplified, of SEQ IDNo. 8:

UAUCCAAUUU GGGUAGACCA GUGGCCUUUA AAGGGAGAGA
AAUUGCAAAG AGCCCAUGAG UUUGUUGAAG AGCAAUUAAA
AGCCAGCCAU AUAGAACCAU CAAAAAGACC UUGGAAUUCA
CCCAUUUUCA UCAUUCCCAA AAAGUCUGGU AAAUAGAGAC
UUUUGCAUGA CUAACAUGCU AUCAAUGCUA AUUUGAAACC
UAUGGGACCC CUUGAGCAGG UGUUCUCCUC CCCUUCAGUG
AUUCCUUGAG AUUGGCAUAU AAUAGUUAUU GGCUUAAAAU
ACUGCAUUUG UACUAUUCCC UUUGCAGAAC AGGACAGAGA
AAAAUUUGUG UUUAUAAUAC CAGCUAUAAA UAACGAAAGG
CCAGCUCCCC GAUUUCACUG GAAAGUGCUU CCUGAAGGGA
UGCUAAACAG UCCUACCAUG UGUCAGUAUC AUGUAAAUCA
AGCUUUGCUC CUCAGUAGAA AAGAAUUUCC UAAUUGCAAG
ACUAUUCAUU UUAUGGAUGA UAUUUUACUA GCAGCCCCAA
UGGAUCCAGC ACUUUUAAGU UUAUGUGCCU CUGUUGUAAA
GAAUACACAG UUAAGAGGUU UAAUCAUAGC ACCUGAAAAA
GUACAGAUGU CCUCUCCUUG GAAAUAUCUU GGGUACAUAC
UAACUUCGUG GUCAGUAAGA CCUCAAAAGG UUAAAUUAAA
UACUAGCAAC UUACACACCU UAAAUGAUUA UCAGAAAUUA
CUAGGCAAUA UUAACUGGCU UUGCCCCACC UUGGGCAUAA
CUACUGAUAA GUUACAGAAC CUGUUUUCUA UCUUAAAAGA
CAAUGCUGCU CUAGACUCUC CCAGGUAUUU AACUCCUGCA
GCACAAAGGG AAAUUGAGGA AGUAGAGCAC

was produced from cloned sequences derived from a region of the pol gene of the HML-5 endogenous retrovirus in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

In a comparable manner, a third RNA sequence to be amplified, of SEQ ID No. 9:

CACCCUUACC CUGCUCAAUG CCAAUAUCCC AUCCCACAGC
AUGCUUUAAA AGGAUUAAAG CCUGUUAUCA CUCGCCUGCU
ACAGCAUGGG CUUCUAGAAC CUAUAAACUC UCUUUUCAAU
UCCCCCAUUU UACCUGUCCA AAAACCGGAU AAGUCUUACA
GACUAGUUCA GAAUCUGCGU CUUAUCAACC AAAUUGUUUU
GCCUAUCCAC CCUGUGGUGC CCAACCUGUA CACUCUUUUG
UCCUCAAUAC CUUCCUCCAC AACUCACUAU UCCGUGCUUG
AUCUUAAAGA UGGUUUUUUC ACUAUUCUCC UGCACUCCUC
GUCCCAGCCU CUCUUUGCUU UCACCUGGAC UGACCCUGAC
ACCCAUCAGU CCCAGCAGCU UACCUGGACU GUGCUGCCGC
AAGGUUUCAG GGACAGCCCU CGUUACUUCA GCCAAGCUCU
UUCUCAUGAU CUACUUUCCU UCCACCCCUC CGCUUCUCAC
CUUAUUCAAU AUAUUGAUGA GCUUCUUCUU UGUAGCCCCU
CCUUUGAAUC UUCUCAAUAA GACACACUUC UGCUCCUUCA
GCAUUUAUUC UCCAAAGGAU AUCGGGUAUC CCCCUCCAAA
GCUCAAAUUU CUUCUCCAUC CGUUACCUAC CUCGGCAUAA
UUCUUCAUAA GAACACACGU GCUCUCCCUG CCGACCGUGU
CUGACUAAUC UCUCAAGCCC CAACCCCUUC UACAAAACAA
CAACUCCUUU CCUUCCUGGG CGUGGUUGGA UACUUUCGCC
UUUGGAUACC UGGUUUUGCC AUCCUAACAA AACCAUUAUA
UAAACUCACA AAAGGAAACU UAGCUGACCC CAUAGAUCCU
AAAUCCUUUC CCCACUCCUC UUUCUG

was produced from cloned sequences derived from a region of the pol gene of the HERV-H endogenous retrovirus in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

In a comparable manner, a fourth RNA sequence to be amplified, of SEQ ID No. 10:

CUGAAGCUCU UAAAGGAUUA CAGGAUAUUG UUAAACAUUU
AAAAGCUCAA GGCUUAGUAA GGAAAUGCAG CAGUCCCUGC
AACACCCCAA UUCUAGGAGU ACAAAAACCA AAUGGUCACU
GGAGACUAGU GCAAGAUCUU AGACUCAUCA AUGAGGCAGU
AAUUCCUCUA UAUCCAGUUG UACCCAACCC CUAUACCCUG
CUUUCUCAAA UACCAGAGGA AGCAGAAUGG UUCAUGGUUC
UGGACCUCAA GGAUGCCUUC UUCUGUUUCC CCUGCACUCU
GACUCCCAGU UUCUGUUUGC CUUUGAGGAU CCCACAGACC
ACACGUCCCA ACUUACAUGG AUGGUCUUGC CACAAGGGUU
UAGGGAUAGC CCUCACCUGU UUGGUCAGGC ACUGGCCCAA
GAUCUAGGCC ACUUCUCAAG UCCAGGCACU UUGGUCCUUC
AGUAUGUGGA UGAUUUACUU UUGGCUACCA GUUCAGAAGC
CUCAUGCCAG CAGGCUACUC UAGAUCUCUU GAACUUUCUA
CCUAAUCAAG GGUACAAGGC AUCUAGGUCA AAGGUGCAGC
UUUGCUUACA GCAGGCUAAA UAUCUAGGCC UAAUCUUAGC
CAGAGGGACC AGGGCCCUCA GCAAGGAAUG AAUACAGCCU
AUACUGGCUU AUCCUUGCCC UAAGACAUUA AAACAGUUGC
AGGGGUUCCU UGGAAUCACC GGCUUUUGCC GACUAUGGAU
CCCUGGAUAC AGUGAGAUAG UCAGGCCCCU CCAUACUCUA
AUCAAGGAGA CCC

was produced from cloned sequences derived from a region of the pol gene of the ERV-9 endogenous retrovirus in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

In a comparable manner, a fifth RNA sequence to be amplified, of SEQ ID No. 11:

GACCCAAGGC CCAACAAGGA CUCCAAAAGA UUGUUAAGGA
CCUAAAAGCC CAAGGCCUAG UAAAACCAUG CAGUAACCCC
UGCAGUACUC CAAUUUUAGG AGUACAGAAA CCCAACAGAC
AGUGGAGGUU AGUGCAAGAU CUCAGGAUUA UCAAUGAGGC
UGUUGUUCCU CUAUAGCCAG CUGUACCUAG CCCUUAUACU
CUGCUUUCCC AAAUACCAGA GGAAGCAGAG UGGUUUACAG
UCCUGGACCU UCAGGAUGCC UUCUUCUGCA UCCCUGUACA
UCCUGACUCU CAAUUCUUGU UUGCCUUUGA AGAUACUUCA
AACCCAACAU CUCAACUCAC CUGGACUUUU UACCCCAAGG
UUCAGGGAUA GUCCCCAUCU AUUUGCCAGG CAUUAGCCCA
AGACUUGAGC CAAUCCUCAU ACCUGGACAC UUGUCUUCGG
UAGGUGGAUG AUUUACUUUU GGCCGCCCAU UCAGAAACCU
UGUGCCAUCA AGCCACCCAA GCGCUCUUCA AUUUCCUCGC
UACCUGUGGC UACAUGGUUU CCAAACCAAA GGCUCAACUC
UGCUCACAGC AGGUUACUUA GGGCUAAAAU UAUCCAAAGG
CACCAGGGCC CUCAGUGAGG AACACAUCCA GCCUAUACUG
GCUUAUCCUC AUCCCAAAAC CCUAAAGCAA CUAAGGGGAU
UCCUUGGCGU AAUAGGUUUC UGCCGAAAAU GGAUUCCCAG
GUAUGGCGAA AUAGCCAGGU CAUUAAAUAC ACUAAUUAAG
GAAACUCAGA AAGCCAAUAC CCAUUUAGUA AGAUGGACAA
CUGAAGUAGA AGUGG

was produced from cloned sequences derived from a region of the pol gene of the HERV-W endogenous retrovirus in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

In a comparable manner, a sixth RNA sequence to be amplified, of SEQ ID No. 12:

AUCAUAAUCC AAUGCCAGUC ACCCUGGAAC ACUCCACUUU
UGCUGGUACA AAAACCAUUG CCUGGACCAG GAUCUGAUGA
GUAUAUACUG GUGCAGGGCU UGCAUGCUGU AAACCAAGCC
ACGGUGACCA UACAUCCAGU AGUACCAAAC CUGUAUACUU
UAAUGGGACU UAUUCUAGCA AGUGCCACCU GGUUUACAGU
CCUGGACUUA AAGGAUGCUU UCCUCUGUCU CUACCUGGCA
CCAGUUAGUC AGCCCAUCUU UGCAUUUUAA UGGGACAAUU
CAGUCACAGG CACAGGGGGA CAGCUCGCCU GGACUAGUCU
CCCACAAGGG UUCAAGAAUU CUCCCACAAU CUUUGGGGAA
GCACUGGCCU CAGACCUCAA GGCAUACACC CCACCAAAUG
ACAACUGCGC CUUGUUGCAG UACAUAGACA ACCCUUCUUU
UGGCAGCCCC AACCCCAAGA GGACUGUAAC UGGGGGAACC
CAGGACCUCC UCCAUCUCUU AUGGGAAAGC AGGGUAUAGA
GUAUCCAAAA AGAAGGCCCA AAUUUGCCAU GAAAAGGUUA
AAUAUUUAGG CUUCAUAGUA AGCCAAGGGG AACGCUGGCU
UGGCCAUGGA UGAAAGCAAG CCAUUUGUGC ACUUCCAACU
CCAACCACCU GGCGCCAAAU AGGGGAAUUC UUAGGGGCAG
CAGGGUUCUG CCAUAUCUGG AUCCCAAAUU UCUCACUUAU
AGCCAGGCCC UUAUAUGAAG CCACAAGGGA GGGUGAAAAG
GAACCCCUCC UCUGAAAGGC UGACCAGAAG AAGGUGUUUA
AACAAAUCAA AGAAGCUCUA ACUCAGGCUC CAGCCUUAGG
ACUGCCAGAU ACUACU

was produced from cloned sequences derived from a region of the pol gene of the HERV-R endogenous retrovirus in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

In a comparable manner, a seventh RNA sequence to be amplified, of SEQ ID No. 13:

UUAAUGGAGU UAUGGCUCAG GUCUGACUUA CAGCAGGUCC
AGUGGGUCCC UGGACUCAUC CUGUGUUCAU UUUCCCAGUG
CCAGAAUGCA UAAUUGGCAU UGUCAUACUU AGAGGCUGGC
AGAACCCCCA CAUUGAUUCU GUGACUGGUA AGGUGAGGGC
UAUUAUGGUG GGAAAGGCCA AAUGGAAGCC AUUGGAGCUG
CCUUUACCUA GGAAAAUAGU AAAUCAAAAA CAUUAUCACC
ACCCUGGAGG GAUUGCAGAG AUUAGUGCCA CCAUCAAGGA
CUUGAAAAAU GCAGGGGUGG CGAUUCCCAU AACAUCCUUG
UUCAACUCUC CUUUUUGGCC UGUGCAGAAG ACAGAUGGAU
CUUGGAGAAU GAGAGUGGAU UAUCAUAAGC UUAACCAAGU
GGUGACUCCA AUUGCAGCUG CUAUACAAGA UGUGGUUUUC
AUUGCUCAAG CAAAUUAAUA CAUCUCCUGG UACCUUGUAU
GCAGCCAUUG ACUUGGCAAA UGGCCUUUUA CCCAUUCCAU
AAGCCCCACC AGAAGCGAUU UGCCUUCAGC UGGCAAGGCC
AGCAAUGUAU CUUUACUGUC CUACUUCAGG GGUAUAUCAA
CUCUCCGGCU UUGUGUCAUA AUCUUAUUCA GAGUGAUCUU
GAUCACUUUU CACUGCCACA AGAUAUCACA CUGGUCCAUU
ACAUUGAUGG CAUUAUGUUG AUUGGAUCCA AUGAGCAAGA
AGUAGCAAAC ACACUGGACU UAUUGGUGAG ACAUUUGCAU
GCCAUAGGAU GGGAAAUAAA UCCAAAUAAA AUUCACGCAC
CCUCUACCUC AGUAAAAUU

was produced from cloned sequences derived from a region of the pol gene of the HERV-L endogenous retrovirus in a Dual promoter vector pCR-II-TOPO (Invitrogen) according to the protocol recommended by the supplier. The transcription was carried out using the Ampliscribe T7 Flash kit (Epicentre) according to the protocol recommended by the supplier.

The amplification was carried out, as described in example 2, in the presence of:
the primer sequence of SEQ ID No. 14 AATTCTAATA CGACTCACTA TAGGGAGACA CATAAAATAT CATCAACATA (0.75 μM) for amplifying the RNA sequence of SEQ ID No. 7,
the primer sequence of SEQ ID No. 15 AATTCTAATA CGACTCACTA TAGGGAGATG CAGAGGAGAT CATCCATGTA (0.75 μM) for amplifying the RNA sequence of SEQ ID No. 1,
the primer sequence of SEQ ID No. 16 AATTCTAATA CGACTCACTA TAGGGAGACT AGTAAAATAT CATCCATAAA (0.75 μM) for amplifying the RNA sequence of SEQ ID No. 8,
the primer sequence of SEQ ID No. 17 AATTCTAATA CGACTCACTA TAGGGAGAAA AGTAGAAGGT CATCAATATA (0.75 μM) for amplifying the RNA sequence of SEQ ID No. 9,
the primer sequence of SEQ ID No. 18 AATTCTAATA CGACTCACTA TAGGGAGAAG TAAATCATCC ACATACTGAA G (0.75 μM) for amplifying the RNA sequence of SEQ ID No. 10,
the primer sequence of SEQ ID No. 19 AATTCTAATA CGACTCACTA TAGGGAGAAA GTAAATCATC CACGTACCGA (0.75 μM) for amplifying the RNA sequence of SEQ No. 11,
the primer sequence of SEQ ID No. 20 AATTCTAATA CGACTCACTA TAGGGAGACC AGCAAAAGGT CATCAACGTA (0.75 μM) for amplifying the RNA sequence of SEQ ID No. 2,
the primer sequence of SEQ ID No. 21 AATTCTAATA CGACTCACTA TAGGGAGACC AAAAGAAGGT TGTCTATGTA (0.75 μM) for amplifying the RNA sequence of SEQ ID No. 12,
the primer sequence of SEQ ID No. 22 AATTCTAATA CGACTCACTA TAGGGAGATC ARCATAATGY CATCAATGTA (0.75 μM) for amplifying the RNA sequence of SEQ ID No. 13.

The RNA transcripts obtained were analyzed on an OLISA chip as described in example 2.

The probe for demonstrating SEQ ID No. 1 was the sequence SEQ ID No. 5: CAACCTGTCAGGGATCAGTTTC.

The probe for demonstrating SEQ ID No. 2 was the sequence SEQ ID No. 6: CTCGAGACCTCCAGAAGTTTCC.

The probe for demonstrating SEQ ID No.7 was the sequence SEQ ID No. 23: CAACCAGTTA GAGACAAGTT TTCA.

The probe for demonstrating SEQ ID No.8 was the sequence SEQ ID No. 24: TTGCTCCCCA GTAGAAAAGA ATT.

The probe for demonstrating SEQ ID No. 9 was the sequence SEQ ID No. 25: GACAGCCCCC ATTACTTCAG TCAAG.

The probe for demonstrating SEQ ID No. 10 was the sequence SEQ ID No. 26: CCAAGATCTA GGCCACTTCT CA.

The probe for demonstrating SEQ ID No. 11 was the sequence SEQ ID No. 27: CATCTATTTG GCCAGGCATT A.

The probe for demonstrating SEQ ID No. 12 was the sequence SEQ ID No. 28: AGACCTCAAG GCATACACCC.

The probe for demonstrating SEQ ID No. 13 was the sequence SEQ ID No. 29: GCTTTGTGTC ATAATCTTAT TCG.

The means and standard deviations are calculated from four independent amplifications, and shown in FIG. 4.

The technique according to the invention makes it possible to amplify transcripts in a mixture (here, up to 9 different ones). It is also noted that a linear relationship exists between the signal measured and the amount of transcripts introduced into the reaction volume over the range tested (from 10⁶ to 10⁸ copies for each transcript). This indicates that this technique makes it possible to simultaneously and specifically amplify several transcripts. Thus, the DNA-chip analysis (or analysis by another method) of amplification products obtained by means of this method could allow quantification of the transcripts of the initial sample.

1. A method for generating transcripts from:
at least one RNA sequence to be amplified comprising a “primer” region and a region of interest, and
an amplification primer comprising a promoter region, and a region capable of hybridizing to said “primer” region of the RNA sequence to be amplified,
said method being carried out at constant temperature, and comprising the following steps:
a) said primer is hybridized with the RNA to be amplified,
b) the primer is extended by means of a reverse transcriptase enzymatic activity in order to generate a complementary deoxyribonucleic acid (cDNA) sequence of the RNA to be amplified,
c) the RNA to be amplified, hybridized to said cDNA, is cleaved by means of an enzyme that has a ribonuclease H activity, so as to obtain fragments of RNA to be amplified, hybridized to said cDNA,
d) the ends of said fragments of RNA to be amplified are extended by means of a reverse transcriptase and strand displacement enzyme, so as to obtain RNA-DNA/DNA hybrids,
e) RNA transcripts are obtained from the RNA-DNA/DNA hybrids formed in step d), by means of an enzyme that has an RNA polymerase activity.

2. The method for generating transcripts as claimed in claim 1, also comprising the following step:
f. a hybridization probe specific for said region of interest is used for detecting the RNA transcripts generated.

3. The method for generating transcripts as claimed in claim 1, according to which steps a) to e) are carried our for a period of time sufficient to obtain a sufficient number of transcripts.

4. The method for generating transcripts as claimed in claim 1, in which the enzyme that has an RNA polymerase activity is a bacteriophage RNA polymerase, preferably the RNA polymerase of the T7 bacteriophage.

5. A method for generating transcripts from:
at least a first RNA sequence to be amplified comprising a “primer” region and a region of interest, and a first amplification primer comprising a promoter region and a region capable of hybridizing to said “primer” region of the first RNA sequence to be amplified,
at least a second RNA sequence to be amplified, different than said first RNA sequence to be amplified, and comprising a “primer” region and a region of interest, and a second amplification primer comprising a promoter region and a region capable of hybridizing to said “primer” region of the second RNA sequence to be amplified,
said method being carried out at constant temperature, and comprising the following steps:
a). said first and second primers are hybridized with said first and second RNAs to be amplified,
b). said first and second primers are extended by means of a reverse transcriptase enzymatic activity in order to generate a first complementary deoxyribonucleic acid (cDNA) sequence of the first RNA to be amplified and a second complementary deoxyribonucleic acid (cDNA) sequence of the second RNA to be amplified,
c). the first and second RNAs to be amplified, hybridized respectively to the first and second cDNAs, are cleaved by means of an enzyme that has a ribonuclease H activity, so as to obtain first fragments of RNA to be amplified, hybridized to the first cDNA, and second fragments of RNA to be amplified, hybridized to the second cDNA,
d). the ends of said first and second fragments of RNA to be amplified are extended by means of a reverse transcriptase and strand displacement enzyme, so as to obtain RNA-DNA/DNA hybrids,
e). transcripts of said first and second RNAs to be amplified are obtained from the RNA-DNA/DNA hybrids formed in step d), by means of an enzyme that has an RNA polymerase activity.

6. The method for generating transcripts as claimed in claim 5, also comprising the following step:
f. a first hybridization probe is used for detecting the transcripts of the first RNA to be amplified and a second hybridization probe is used for detecting the transcripts of the second RNA to be amplified.

7. The method for generating transcripts as claimed in claim 5, according to which steps a) to e) are carried out for a period of time sufficient to obtain a sufficient number of transcripts.

8. The method for generating transcripts as claimed in claim 5, in which the enzyme that has an RNA polymerase activity is a bacteriophage RNA polymerase, preferably the RNA polymerase of the T7 bacteriophage.

9. The method for generating transcripts as claimed in claim 2, according to which steps a) to e) are carried our for a period of time sufficient to obtain a sufficient number of transcripts.

10. The method for generating transcripts as claimed in claim 2, in which the enzyme that has an RNA polymerase activity is a bacteriophage RNA polymerase, preferably the RNA polymerase of the T7 bacteriophage.

11. The method for generating transcripts as claimed in claim 3, in which the enzyme that has an RNA polymerase activity is a bacteriophage RNA polymerase, preferably the RNA polymerase of the T7 bacteriophage.

12. The method for generating transcripts as claimed in claim 6, according to which steps a) to e) are carried out for a period of time sufficient to obtain a sufficient number of transcripts.

13. The method for generating transcripts as claimed in claim 6, in which the enzyme that has an RNA polymerase activity is a bacteriophage RNA polymerase, preferably the RNA polymerase of the T7 bacteriophage.

14. The method for generating transcripts as claimed in claim 7, in which the enzyme that has an RNA polymerase activity is a bacteriophage RNA polymerase, preferably the RNA polymerase of the T7 bacteriophage.
